(19) United States
(12) Patent Application Publication
(10) Pub. No.: US 2010/0152267 A1
(43) Pub. Date: Jun. 17, 2010

(54) NOVEL RIFAMYCIN 3,4-(3-SUBSTITUTED AMINOMETHYL) FUSED PYRROLO DERIVATIVES

(76) Inventors: Mark J. MACIELAG, Branchburg, NJ (US); Manomi A. Tennakoon, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/611,316

(22) Filed: Nov. 3, 2009

Related U.S. Application Data
(60) Provisional application No. 61/121,773, filed on Dec. 11, 2008.

(51) Int. Cl. A61K 31/407; C07D 498/06

(52) U.S. Cl. 514/410; 540/457

(57) ABSTRACT

The present invention is directed to novel 3,4-(3′-substituted aminomethyl) fused pyrrolo rifamyacin derivatives, pharmaceutical compositions containing them and the use of said derivatives and pharmaceutical compositions as antimicrobial agents against pathogenic microorganisms, particularly against resistant microbes.

### CROSS-REFERENCE TO RELATED APPLICATIONS

The present application claims the benefits of the filing of U.S. Provisional Application No. 61/121,773 filed Dec. 11, 2008. The complete disclosures of the aforementioned related patent applications are hereby incorporated herein by reference for all purposes.

### FIELD OF THE INVENTION

The present invention is directed to novel 3,4-(3′-substituted aminomethyl) fused pyrrolo rifamyacin derivatives, pharmaceutical compositions containing them and the use of said derivatives and pharmaceutical compositions as antimicrobial agents against pathogenic microorganisms, particularly against resistant microbes.

### BACKGROUND OF THE INVENTION

The chemical and medical literature describes compounds that are said to be antimicrobial, i.e., capable of destroying or suppressing the growth or reproduction of microorganisms, such as bacteria. For example, such antibacterial agents are described in Antibiotics, Chemotherapeutics, and Antibacterial Agents for Disease Control (M. Greyson, editor, 1982), E. Gale et al., The Molecular Basis of Antibiotic Action 2d edition (1981), Recent Research Developments in Antimicrobial Agents & Chemotherapy (S. G. Pandalai, Editor, 2001), Quinolone Antimicrobial Agents (John S Wolfson., David C Hooper, Editors, 1989), and F. O'Grady, H. P. Lambert, R. G. Finch, D. Greenwood, Martin Dedicoat, “Antibiotic and Chemotherapy, 7th edn.” (1997).

The mechanisms of action of these antibacterial agents vary. However, they are generally believed to function in one or more ways: by inhibiting cell wall synthesis or repair; by altering cell wall permeability; by inhibiting protein synthesis; or by inhibiting the synthesis of nucleic acids. For example, beta-lactam antibacterial agents act through inhibiting essential penicillin binding proteins (PBPs) in bacteria, which are responsible for cell wall synthesis. As another example, quinolones act, at least in part by inhibiting synthesis of DNA, thus preventing the cell from replicating.

The pharmacological characteristics of antimicrobial agents, and their suitability for any given clinical use, vary. For example, the classes of antimicrobial agents (and members within a class) may vary in 1) their relative efficacy against different types of microorganisms, 2) their susceptibility to development of microbial resistance and 3) their pharmacological characteristics such as their bioavailability and biodistribution. Accordingly, selection of an appropriate antimicrobial agent in a given clinical situation requires analysis of many factors, including the type of organism involved, the desired method of administration, the location of the infection to be treated and other considerations.

However, many such attempts to produce improved antimicrobial agents yield equivocal results. Indeed, few antimicrobial agents are produced that are truly clinically acceptable in terms of their spectrum of antimicrobial activity, avoidance of microbial resistance, and pharmacology. Thus there is a continuing need for broad-spectrum antimicrobial agents, which are effective against resistant microbes.

Examples of bacterial infections resistant to antibiotic therapy have been reported in the past; they are now a significant threat to public health in the developed world. The development of microbial resistance (perhaps as a result of the intense use of antibacterial agents over extended periods of time) is of increasing concern in medical science. “Resistance” can be defined as existence of organisms, within a population of a given microbial species, that are less susceptible to the action of a given antimicrobial agent. This resistance is of particular concern in environments such as hospitals and nursing homes, where relatively high rates of infection and intense use of antibacterial agents are common. See, e.g., W. Sanders, Jr. et al., “Inducible Beta-lactamases: Clinical and Epidemiologic Implications for the Use of Newer Cephalosporins”, Review of Infectious Diseases, p. 830 (1988).

Pathogenic bacteria are known to acquire resistance via several distinct mechanisms including inactivation of the antibiotic by bacterial enzymes (e.g., β-lactamases hydrolyzing penicillin and cephalosporins); removal of the antibiotic using efflux pumps; modification of the target of the antibiotic via mutation and genetic recombination (e.g., penicillin-resistance in Neiserria gonorrhoeae); and acquisition of a readily transferable gene from an external source to create a resistant target (e.g., methicillin-resistance in Staphylococcus aureus). There are certain Gram-positive pathogens, such as vancomycin-resistant Enterococcus faecium, which are resistant to most commercially available antibiotics.

Hence existing antibacterial agents have limited capacity in overcoming the threat of resistance. Thus it would be advantageous to provide new antibacterial agents that can be used against resistant microbes.

### SUMMARY OF THE INVENTION

The present invention is directed to compounds of formula (I).

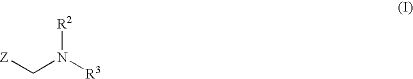

wherein

Z is selected from the group consisting of

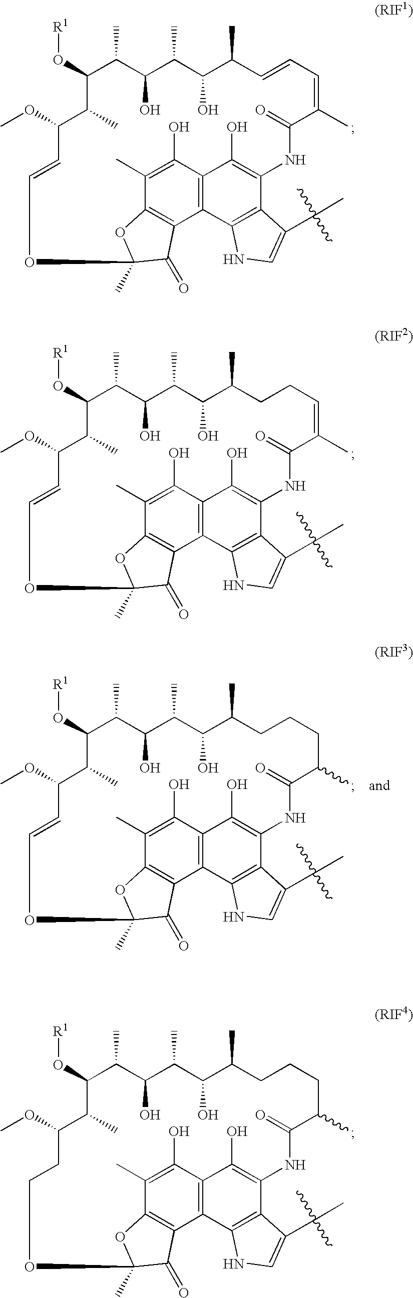

R¹ is selected from the group consisting of hydrogen and acyl;

R² and R³ are each independently selected from the group consisting of hydrogen, C₁₋₄alkyl and —(C₁₋₄alkyl)-(Ring A)-(Ring B);

wherein (Ring A) is selected from the group consisting of aryl and heteroaryl; wherein the aryl or heteroaryl is optionally substituted with one or more substituents independently selected from the group consisting of halogen, C₁₋₄alkyl, C₁₋₄alkoxy, halogenated C₁₋₄alkyl and halogenated C₁₋₄alkoxy;

wherein (Ring B) is selected from the group consisting of aryl, heteroaryl and heterocycloalkyl; wherein the aryl, heteroaryl or heterocycloalkyl is optionally substituted with one or more substituents independently selected from the group consisting of halogen, C₁₋₄alkyl, C₁₋₄alkoxy, halogenated C₁₋₄alkyl and halogenated C₁₋₄alkoxy;

alternatively R² and R³ are taken together with the nitrogen atom to which they are bound to form a 5 to 7 membered, saturated, nitrogen containing ring; wherein the 5 to 7 membered, saturated nitrogen containing ring is optionally substituted with one or more substituents independently selected from the group consisting of halogen, C₁₋₄alkyl, C₁₋₄alkoxy, halogenated C₁₋₄alkyl and halogenated C₁₋₄alkoxy;

and pharmaceutically acceptable salts, esters and prodrugs thereof.

Illustrative of the invention is a pharmaceutical composition comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

It has been found that the compounds of this invention, and compositions containing these compounds, are effective antimicrobial agents against a broad range of pathogenic microorganisms with advantages of activity against resistant microbes.

Accordingly, the present invention is also directed to a method of treating a subject having a condition caused by or contributed to by bacterial infection, which comprises administering to said mammal a therapeutically effective amount of a compound of formula (I).

The present invention is further directed to a method of preventing a subject from suffering from a condition caused by or contributed to by bacterial infection, which comprises administering to the subject a prophylactically effective dose of the pharmaceutical composition of a compound of formula (I).

The present invention is further directed to the use of a compound of formula (I) for the preparation of a medicament for treating and/or preventing a condition caused by or contributed to by bacterial infection, in a subject in need thereof. In an embodiment, the present invention is directed to the use of a compound of formula (I) for the preparation of a medicament for treating and/or preventing a condition caused by or contributed to by bacterial infection associated with a drug resistant bacteria, in a subject in need thereof.

### DETAILED DESCRIPTION OF THE INVENTION

The present invention is directed to compounds of formula (I)

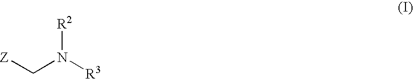

wherein Z, R², and R³ areas herein defined; and pharmaceutically acceptable salts, esters and prodrugs thereof. The compounds of formula (I) are useful as antimicrobial agents against pathogenic microorganisms, preferably, resistant microbes.

In an embodiment of the present invention, Z is selected from the group consisting of (RIF¹), (RIF²), (RIF³), and (RIF⁴). In another embodiment of the present invention, Z is selected from the group consisting of (RIF¹) and (RIF²). In another embodiment of the present invention, Z is (RIF¹).

In another embodiment of the present invention, R¹ is acyl, preferably, R¹ is selected from the group consisting of —C(O)—(C₁₋₄alkyl), preferably —C(O)—(C₁₋₂alkyl). In another embodiment of the present invention, R¹ is —C(O)—CH₃. In another embodiment of the present invention, R¹ is selected form the group consisting of hydrogen and —C(O)—CH₃.

In an embodiment of the present invention, R² and R³ are each independently selected from the group consisting of hydrogen, C₁₋₄alkyl and —(C₁₋₄alkyl)-(Ring A)-(Ring B); wherein (Ring A) is selected from the group consisting of phenyl and 5 to 6 membered heteroaryl; wherein the aryl or 5 to 6 membered heteroaryl is optionally substituted with one to three substituents independently selected from the group consisting of halogen, C₁₋₄alkyl, C₁₋₄alkoxy, halogenated C₁₋₄alkyl and halogenated C₁₋₄alkoxy; and wherein (Ring B) is selected from the group consisting of phenyl, 5 to 6 membered heteroaryl and 5 to 6 membered heterocycloalkyl; wherein the phenyl, 5 to 6 membered heteroaryl or 5 to 6 membered heterocycloalkyl is optionally substituted with one to three substituents independently selected from the group consisting of halogen, C₁₋₄alkyl, C₁₋₄alkoxy, halogenated C₁₋₄alkyl and halogenated C₁₋₄alkoxy.

In another embodiment of the present invention, R² and R³ are each independently selected from the group consisting of hydrogen, C₁₋₂alkyl and —(C₁₋₄alkyl)-(Ring A)-(Ring B); wherein (Ring A) is phenyl; and wherein (Ring B) is selected from the group consisting of phenyl, 5 to 6 membered heteroaryl and 5 to 6 membered heterocycloalkyl; wherein the phenyl, 5 to 6 membered heteroaryl or 5 to 6 membered heterocycloalkyl is optionally substituted with one to two substituents independently selected from the group consisting of halogen, C₁₋₄alkyl, C₁₋₄alkoxy, fluorinated C₁₋₂alkyl and fluorinated C₁₋₂alkoxy.

In another embodiment of the present invention, R² and R³ are each independently selected from the group consisting of hydrogen, methyl and 4-(4-methyl-piperazinyl)-benzyl. In another embodiment of the present invention, R² and R³ are each independently selected from the group consisting of hydrogen and 4-(4-methyl-piperazinyl)-benzyl.

In an embodiment of the present invention, R² and R³ are taken together with the nitrogen atom to which they are bound to form a 5 to 6 membered, saturated, nitrogen containing ring; wherein the 5 to 6 membered, saturated nitrogen containing ring is optionally substituted with one to three substituents independently selected from the group consisting of halogen, C₁₋₄alkyl, C₁₋₄alkoxy, halogenated C₁₋₄alkyl and halogenated C₁₋₄alkoxy. In another embodiment of the present invention, R² and R³ are taken together with the nitrogen atom to which they are bound to form a 5 to 6 membered, saturated, nitrogen containing ring; wherein the 5 to 6 membered, saturated nitrogen containing ring is optionally substituted with one to two substituents independently selected from the group consisting of halogen, C₁₋₄alkyl, C₁₋₄alkoxy, fluorinated C₁₋₂alkyl and fluorinated C₁₋₂alkoxy. In another embodiment of the present invention, R² and R³ are taken together with the nitrogen atom to which they are bound to form 1-piperidinyl.

Additional embodiments of the present invention, include those wherein the substituents selected for one or more of the variables defined herein (i.e. Z, R¹, R² and R³) are independently selected to be any individual substituent or any subset of substituents selected from the complete list as defined herein.

Representative compounds of the present invention are as listed in Table 1, below. In another embodiment of the present invention is any single compound or subset of compounds selected from the representative compounds listed in Table 1.

TABLE 1
Representative Compounds of Formula (I)

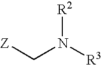

| ID No. | Z | R¹ | R² | R³ |
|---|---|---|---|---|
| 1 | Rif¹ | —C(O)—CH₃ | H | H |
| 3 | Rif¹ | —C(O)—CH₃ | methyl | 4-(4-methyl- |
| | | | | piperazinyl)-benzyl |
| 4 | Rif¹ | —C(O)—CH₃ | H | 4-(4-methyl- |
| | | | | piperazinyl)-benzyl |
| 5 | Rif¹ | —C(O)—CH₃ | 4-(4-methyl- | 4-(4-methyl- |
| | | | piperazinyl)- | piperazinyl)-benzyl |
| | | | benzyl | |

| | | | R² + R³ taken together with the |
|---|---|---|---|
| ID No. | Z | R¹ | nitrogen atom |
| 2 | Rif¹ | —C(O)—CH₃ | 1-piperidinyl |

In an embodiment, the present invention is directed to compounds of formula (I) whose MIC (minimum inhibitory concentration) against strain A as measured according to the procedure described in Example 5 is less than or equal to about 8 μg/mL, preferably less than or equal to about 2 μg/mL, more preferably less than or equal to about 1 μg/mL. In an embodiment, the present invention is directed to compounds of formula (I) whose MIC (minimum inhibitory concentration) against strain B as measured according to the procedure described in Example 5 is less than or equal to about 4 μg/mL, preferably less than or equal to about 1 μg/mL, more preferably less than or equal to about 0.25 μg/mL. In an embodiment, the present invention is directed to compounds of formula (I) whose MIC (minimum inhibitory concentration) against strain C as measured according to the procedure described in Example 5 is less than or equal to about 4 μg/mL, preferably less than or equal to about 1 μg/mL, more preferably less than or equal to about 0.12 μg/mL. In an embodiment, the present invention is directed to compounds of formula (I) whose MIC (minimum inhibitory concentration) against strain D as measured according to the procedure described in Example 5 is less than or equal to about 4 μg/mL, preferably less than or equal to about 0.5 μg/mL, more preferably less than or equal to about 0.25 μg/mL.

As used herein, the terms “halo” or “halogen” shall mean fluoro, chloro, bromo or iodo; preferably fluoro, chloro or bromo, more preferably fluoro.

As used herein, the term “alkyl” shall mean a saturated, straight or branched hydrocarbon chain having 1 to 8 carbons. For example, alkyl radicals include methyl, ethyl, propyl, isopropyl, butyl, isobutyl, sec-butyl, t-butyl, pentyl and the like.

The term “halogenated C₁₋₄alkyl” shall mean any C₁₋₄alkyl group as defined above substituted with one to five halogen atoms, preferably with at least one halogen atom, preferably substituted with a least one fluoro atom. Suitable examples include but are not limited to —CF₃, —CH₂—CF₃, —CF₂—CF₂—CF₂—CF₃, and the like. Similarly, the term “fluorinated C₁₋₄alkyl” shall mean any C₁₋₄alkyl group as defined above substituted with one to five fluoro atoms, preferably with at least one fluoro atom. Suitable examples include but are not limited to —CF₃, —CH₂—CF₃, —CF₂—CF₂—CF₂—CF₃, and the like.

The term “alkoxy” shall denote an oxygen ether radical of the above described straight or branched chain alkyl groups (i.e. a group of the formula —O-alkyl). For example, methoxy, ethoxy, n-propoxy, sec-butoxy, t-butoxy, n-hexyloxy and the like. The term “halogenated C₁₋₄alkoxy” shall mean any C₁₋₄ alkoxy group as defined above substituted with one to five halogen atoms, preferably with at least one halogen atom, preferably substituted with a least one fluoro atom. Suitable examples include but are not limited to —OCF₃, —OCHF₂, —OCH₂—CF₃, —OCF₂—CF₂—CF₂—CF₃, and the like. Similarly, the term “fluorinated C₁₋₄alkoxy” shall mean any C₁₋₄alkoxy group as defined above substituted with one to five fluoro atoms, preferably with at least one fluoro atom. Suitable examples include but are not limited to —OCF₃, —OCHF₂, —OCH₂—CF₃, —OCF₂—CF₂—CF₂—CF₃, and the like.

As used herein, the prefix “C_{x-y}” wherein x and y are numbers shall denote the number of carbon atoms present in a particular functional group. For example, the term “C₁₋₄alkyl” denotes any straight or branched chain alkyl as herein defined of between 1 and 4 carbon atoms, inclusive. Similarly, the term “C₁₋₄alkoxy” shall denote an alkoxy group of between 1 and 4 carbon atoms inclusive.

The term “acyl” shall mean an organic radical of the formula —C(O)—(C₁₋₆alkyl) wherein the C₁₋₆alkyl is any straight or branched chain alkyl as herein defined; the acyl group may be derived from an organic acid by removal of the hydroxyl. Suitable examples include but are not limited to acetyl, propionyl and the like. Preferably, the acyl group is —C(O)—(C₁₋₄alkyl), more preferably, the acyl is —C(O)—CH₃.

The term “aryl” shall refer to carbocylic aromatic groups such as phenyl, naphthyl, and the like, optionally substituted with one or more substituents as herein defined.

The term “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure. Unless otherwise noted, the heteroaryl group may be optionally substituted with one or more substituents as herein defined.

Examples of suitable heteroaryl groups include, but are not limited to, pyrrolyl, furyl, thienyl, oxazolyl, imidazolyl, pyrazolyl, isoxazolyl, isothiazolyl, thiazolyl, triazolyl, thiadiazolyl, pyridyl, pyridazinyl, pyrimidinyl, pyrazinyl, pyranyl, furazanyl, indolizinyl, indolyl, isoindolyl, indazolyl, benzofuryl, benzothienyl, benzimidazolyl, benzothiazolyl, purinyl, quinolinyl, isoquinolinyl, cinnolinyl, phthalazinyl, quinazolinyl, quinoxalinyl, naphthyridinyl, pteridinyl, and the like.

As used herein, the term “heterocycloalkyl” shall denote any 5 to 7 membered monocyclic, saturated or partially unsaturated ring structure containing at least one heteroatom selected from the group consisting of O, N and S, optionally containing one to three additional heteroatoms independently selected from the group consisting of O, N and S; or a 9 to 10 membered saturated, partially unsaturated or partially aromatic bicyclic ring system containing at least one heteroatom selected from the group consisting of O, N and S, optionally containing one to four additional heteroatoms independently selected from the group consisting of O, N and S. The heterocycloalkyl group may be attached at any heteroatom or carbon atom of the ring such that the result is a stable structure. Preferably, the heterocycloalkyl is any 5 to 7 membered monocyclic, saturated or partially unsaturated ring structure containing at least one heteroatom selected from the group consisting of O, N and S, optionally containing one to three additional heteroatoms independently selected from the group consisting of O, N and S.

Examples of suitable heterocycloalkyl groups include, but are not limited to, pyrrolinyl, pyrrolidinyl, dioxolanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, and the like. Preferred heterocycloalkyl groups include, piperidinyl, piperazinyl, pyrrolidinyl and morpholinyl.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein. One skilled in the art will further recognize that substituents may be bound to any of the atoms of a particular group (including, but not limited to C, N or S atoms), provided that the substitution results in a stable structure and does not violate valence rules.

When a particular group is “substituted” (e.g., alkyl, cycloalkyl, aryl, heteroaryl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center. Some of the compounds of the present invention may have trans and cis isomers. In addition, where the processes for the preparation of the compounds according to the invention give rise to a mixture of stereoisomers, these isomers may be separated by conventional techniques such as preparative chromatography. The compounds may be prepared as a single enantiomer, in racemic form, or as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at a diastereomeric excess of greater than or equal to about 80%, more preferably, at a diastereomeric excess of greater than or equal to about 90%, more preferably still, at a diastereomeric excess of greater than or equal to about 95%, more preferably still, at a diastereomeric excess of greater than or equal to about 98%, most preferably, at a diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkyl-aminocarbonyl-C₁-C₆alkyl” substituent refers to a group of the formula

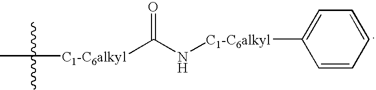

Abbreviations used in the specification, particularly the Schemes and Examples, are as follows

DCE=Dichloroethane

DCM=Dichloromethane

DIPEA=Diisopropylethylamine

EtOAc=Ethyl Acetate

HPLC=High Performance Liquid Chromatography

MeCN=Acetonitrile

MeOH=Methanol

MIC=Minimum Inhibitory Concentration

MPLC=Medium Pressure Liquid Chromatography

TEA=Triethylamine

TFA=Trifluoroacetic acid

THF=Tetrahydrofuran

As used herein, particularly in the scheme which follow herein, (RIF⁵), (RIF⁶), (RIF⁷), (RIF⁸), (RIF⁹), (RIF¹⁰), (RIF¹¹) and (RIF¹²) shall denote rifamyacin derivative substituents of the following structural formulas:

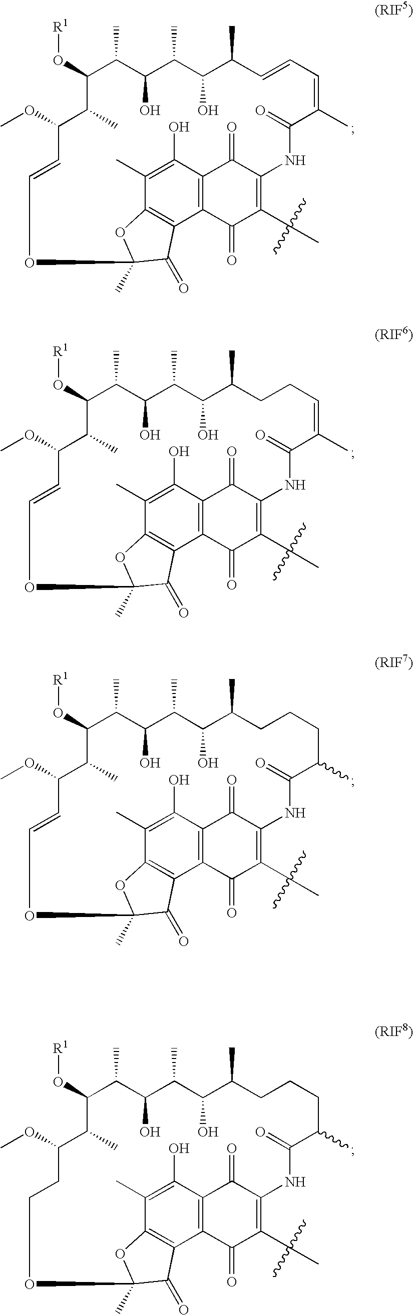

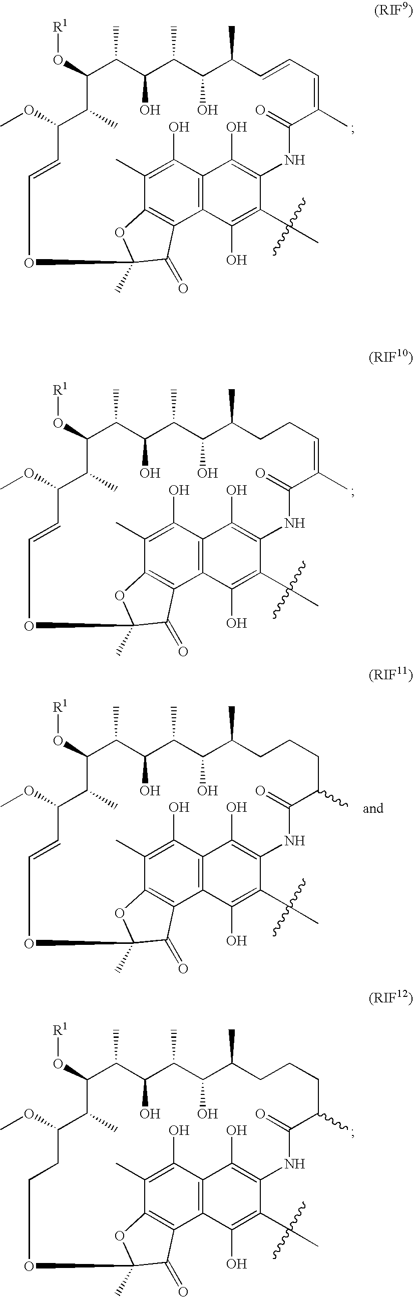

wherein R¹ is as previously defined.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “prophylactically effective amount” as used herein, means that amount of active compound or pharmaceutical agent that prevents the development of a condition, symptoms or manifestations thereof associated with bacterial infection. Thus it elicits the biological or medicinal response in a tissue system, animal or human that is being sought by a researcher, veterinarian, medical doctor or other clinician, which includes alleviation of the symptoms of the disease or disorder being treated.

The term “drug-resistant” or “drug-resistance” refers to the characteristics of a microbe to survive in the presence of a currently available antimicrobial agent such as an antibiotic at its routine, effective concentration.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same or different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

As used herein, unless otherwise noted, the term “leaving group” shall mean a charged or uncharged atom or group which departs during a substitution or displacement reaction. Suitable examples include, but are not limited to, Br, Cl, I, triflate, tosylate, and the like.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laureate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids that may be used in the preparation of pharmaceutically acceptable salts include, but are not limited to, the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases that may be used in the preparation of pharmaceutically acceptable salts include, but are not limited to, the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine and zinc hydroxide.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

As used herein, unless otherwise noted, the term “isolated form” shall mean that the compound is present in a form which is separate from any solid mixture with another compound(s), solvent system or biological environment. In an embodiment, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment, the compound of formula (I) is present as a form which is substantially free of corresponding salt forms.

In making the compounds of the invention, the order of synthetic steps may be varied to increase the yield of desired product. In addition, the skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

Additionally, the skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. Examples of these manipulations can be found for example in T. Greene, Protecting Groups in Organic Synthesis.

Starting materials used in preparing the compounds of the present invention are known, made by published synthetic methods or available from commercial vendors.

Compounds of formula (I) may be prepared according to the process outlined in Scheme 1, below.

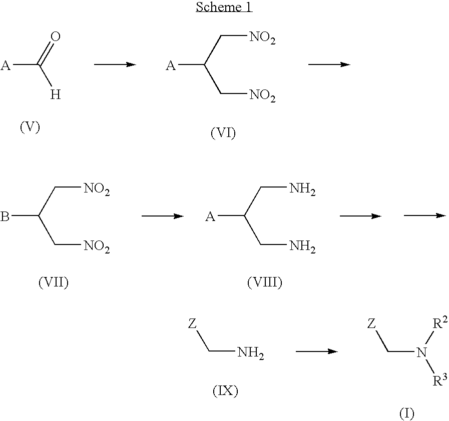

Accordingly, a suitably substituted compound of formula (V), a known compound or compound prepared by known methods, wherein A is selected from the group consisting of (RIF⁹), (RIF¹⁰), (RIF¹¹) and (RIF¹²), a known compound or compound prepared by known methods, is reacted with nitromethane, a known compound; wherein the nitromethane is preferably present in an excess of about 50 to about 100 equivalents, for example about 70 equivalents; in the presence of an organic base such as TEA, DIPEA, pyridine, and the like; neat (i.e. wherein the nitromethane acts as the solvent); preferably at a temperature in the range of from about −20° C. to about 25° C.; preferably for a period of time in the range of from about 24 hours to about 72 hours; to yield the corresponding compound of formula (VI), which compound is optionally not isolated.

The compound of formula (VI) is reacted with a suitably selected oxidizing agent such as manganese dioxide, potassium ferricyanide, and the like, preferably, potassium ferricyanide; in an organic solvent or mixture thereof such as DCM, DCE, water/THF, and the like; preferably at a temperature in the range of from about 0° C. to about 60° C.; preferably for a period of time in the range of from about 1 hour to about 24 hours; to yield the corresponding compound of formula (VII), wherein B is selected from the group consisting of (RIF⁵), (RIF⁶), (RIF⁷) and (RIF⁸).

The compound of formula (VII) is reacted with a suitably selected metal such as indium, zinc, and the like; in the presence of an acid such as HCl, H₂SO₄, TFA, and the like; in an inert solvent or mixture of solvents such as DCM, THF, water/THF, and the like; preferably at a temperature in the range of from about 0° C. to about 60° C.; preferably for a period of time in the range of form about 1 hour to about 24 hours; to yield the corresponding compound of formula (VIII).

The compound of formula (VIII) is reacted in a two step process, to yield the corresponding compound of formula (IX). More particularly, the compound of formula (VIII) is reacted with a suitably selected oxidizing agent such as manganese dioxide, potassium ferricyanide, and the like, preferably, potassium ferricyanide; in an organic solvent or mixture thereof such as DCM, DCE, water/THF, and the like; preferably at a temperature in the range of from about 0° C. to about 60° C.; preferably for a period of time in the range of from about 1 hour to about 24 hours;

and the resulting product mixture is then reacted with a suitably selected reducing agent such as ascorbic acid, and the like; in a solvent or mixture of solvents such as THF/water, acetonitrile/water, methanol/water, and the like; preferably at a temperature in the range of from about 0° C. to about 60° C.; preferably for a period of time in the range of from about 1 hour to about 24 hours; to yield the corresponding compound of formula (IX).

The compound of formula (IX) is reacted with one or two suitably selected aldehyde(s) (more particularly, aldehydes of the desired R² and/or R³ groups), known compound(s) or compound(s) prepared by known methods, in the presence of a suitably selected reducing agent such as sodium triacetoxyborohydride, sodium cyanoborohydride, and the like; in an organic solvent such as DCM, DCE, methanol, and the like; preferably at a temperature in the range of from about −10° C. to about 25° C.; to yield the corresponding compound of formula (I).

One skilled in the art will recognize that wherein the compound of formula (I) R² and R³ are the same, and are other than hydrogen, then the compound of formula (IX) is preferably reacted with about 2 or more molar equivalents of the aldehyde of the desired substituent. One skilled in the art will further recognize that wherein R² and R³ are different and are each other than hydrogen, then the compound of formula (IX) is reacted with two suitably substituted aldehydes of the desired substituents, in a sequential manner, attaching the R² substituent group, followed by attachment of the R³ substituent group; or alternatively in the opposite sequential order, attaching the R³ substituent group, followed by attachment of the R² substituent group.

One skilled in the art will further recognize that wherein R² and R³ are taken together with the nitrogen atom to which they are bound to form an optionally substituted 5 to 7 membered, saturated, nitrogen containing ring, the compound of formula (IX) is reacted with a suitably selected di-aldehyde, under the conditions as described above. As an illustration, Example 2, which follows herein, describes the synthesis of a compound of formula (I) wherein R² and R³ are taken together with the nitrogen atom to which they are bound to form 1-piperidinyl.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gel caps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-1000 mg or any range therein, and may be given at a dosage of from about 0.01-500 mg/kg/day, or any range therein, preferably from about 1 to about 100 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1000 mg, or any range therein, of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations, which generally contain suitable preservatives, are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholine.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxy-ethylaspartamidephenol, or polyethyleneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending on the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment with antimicrobial agents is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any range therein. Preferably, the range is from about 0.1 to about 50 mg/kg of body weight per day, or any range therein. More preferably, from about 0.5 to about 25 mg/kg of body weight per day, or any range therein. The compounds may be administered on a regimen of 1 to 4 times per day.

Optimal dosages to be administered may be readily determined by those skilled in the art, and will vary with the particular compound used, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose in the range of and efficacy trials, in healthy subjects and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples that follow, some synthesis products are listed as having been isolated as a “residue”. It will be understood by one of ordinary skill in the art that the term “residue” does not limit the physical state in which the product was isolated and may include, for example, a solid, an oil, a foam, a gum, a syrup, and the like.

### Example 1

### Compound #1

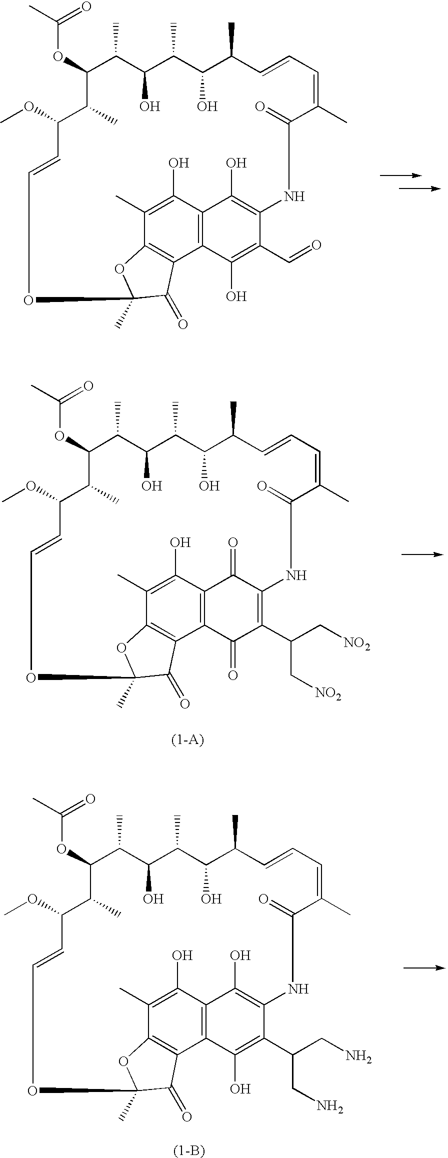

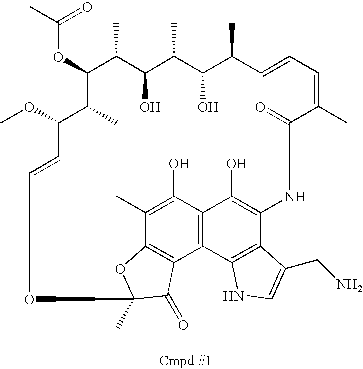

### STEP A:

To a solution of 3-formylrifamycin SV (9.0 g, 12.30 mmol) in nitromethane (50 mL) was added triethylamine (17.4 mL, 123.0 mmol) under nitrogen at room temperature and the resulting mixture stirred at room temperature overnight. The resulting mixture was then concentrated in vacuo, the residue partitioned between EtOAc and 5% aqueous NaH₂PO₄ (˜pH-4), dried with Na₂SO₄, and concentrated in vacuo. To the resulting residue in DCM (50 mL) was added 3\acute{Å} powdered molecular sieves and manganese dioxide (9.00 g, 103.5 mmol) and the resulting mixture was stirred at room temperature for 4 h, filtered through a bed of CELITE®, and concentrated in vacuo. The resulting residue was purified by MPLC (SiO₂, 1-8% gradient elution, MeOH % in DCM) to yield compound (1-A) as a residue.

MS 826 (M−1)⁻

¹H NMR (300 MHz, CDCl₃): δ 8.51 (s, 1H), 6.89 (dd, 1H), 6.53 (d, 1H), 6.24 (dd, 1H), 6.07 (dd, 1H), 5.13-4.91 (m, 5H), 4.86 (d, 1H), 4.25-4.13 (m, 1H), 3.93-3.81 (m, 3H), 3.50-3.48 (m, 1H), 3.10 (s, 3H), 3.08-3.00 (m, 1H), 2.45-2.28 (m, 1H), 2.32 (s, 3H), 2.06-2.04 (m, 6H), 1.87-1.76 (m, 6H), 1.03 (d, 3H), 0.91 (d, 3H), 0.71 (d, 3H), and 0.02 (d, 3H).

### STEP B:

To a solution of the residue prepared in STEP A above (400 mg, 0.48 mmol) in THF (2.6 mL) and water (0.9 mL) was added indium metal powder (444 mg, 3.86 mmol) followed by concentrated HCl (0.48 mL, 5.76 mmol). The resulting mixture was stirred at room temperature for 4 h, filtered and concentrated in vacuo. The resulting residue was purified by HPLC (C-18, 30-100% gradient elution, MeCN/H₂O with 0.1% TFA) to yield compound (1-B) as a residue.

MS 770 (M+1)⁺

### STEP C:

To a solution of the residue prepared in STEP B above (468 mg, 0.47 mmol) in THF (2 mL) was added a solution of potassium ferricyanide (771 mg, 2.30 mmol) in water (1 mL). The resulting mixture was stirred at room temperature for 2 h, partitioned between EtOAc and a solution of 5% Na₂HPO₄, dried with Na₂SO₄, and concentrated in vacuo. The resulting residue was dissolved in MeOH (10 mL)/water (2 mL) and ascorbic acid (404 mg, 2.30 mmol) was added. The resulting mixture was stirred at room temperature for 2 h, partitioned between EtOAc and a solution of 5% Na₂HPO₄, dried with Na₂SO₄, and concentrated in vacuo to yield the title compound. Compound #1 as prepared, was used in the synthesis of additional compounds, as described in Example 2-4 below, without further purification.

MS 748 (M−1)⁻

### Example 2

### Compound #2

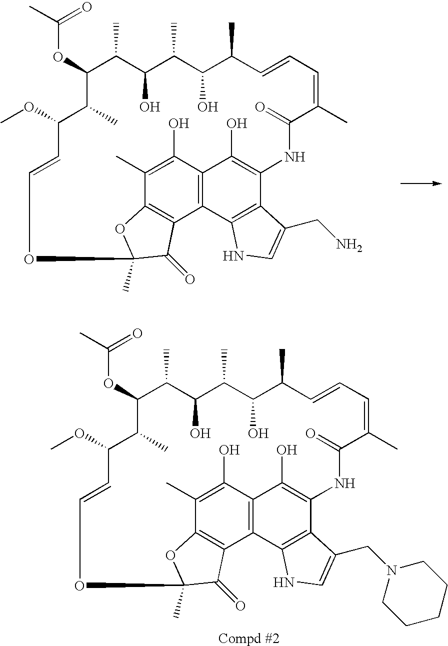

To a solution of Compound #1, prepared as in Example 1 (50 mg, 0.07 mmol) in DCE (2 mL) was added acetic acid (3 drops), 50% aqueous glutaraldehyde (10 μL, 0.1 mmol), and sodium triacetoxyborohydride (106 mg, 0.50 mmol). The resulting mixture was stirred at room temperature for 48 h, partitioned between saturated aqueous sodium bicarbonate and ethyl acetate, dried with sodium sulfate, and concentrated in vacuo. The resulting residue was purified by HPLC (C-18, MeCN/H₂O, gradient elution) to yield the title compound.

MS 818 (M+1)⁺

### Example 3

### Compound #3

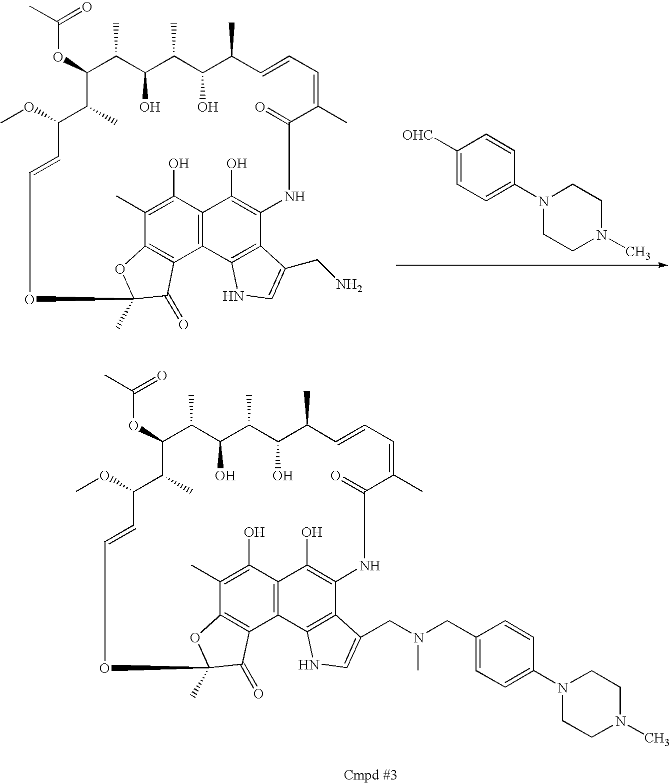

To a solution of Compound #1, prepared as in Example 1 (50 mg, 0.07 mmol) in DCE (0.35 mL) was added acetic acid (0.03 mL, 0.56 mmol), 4-(4-methyl-piperazin-1-yl)-benzaldehyde (17 mg, 0.09 mmol), and sodium triacetoxyborohydride (56 mg, 0.26 mmol). The resulting mixture was stirred at room temperature for 48 h, partitioned between saturated aqueous sodium bicarbonate and ethyl acetate, dried with sodium sulfate, and concentrated in vacuo to yield a residue.

To this residue in DCE (0.35 mL) was added acetic acid (0.03 mL, 0.56 mmol), 37% aqueous formaldehyde (25 μL), and sodium triacetoxyborohydride (56 mg, 0.26 mmol). The resulting mixture was stirred at room temperature for 24 h, partitioned between saturated aqueous sodium bicarbonate and ethyl acetate, dried with sodium sulfate, and concentrated in vacuo. The resulting residue was purified by HPLC (C-18, MeCN/H₂O, gradient elution) to yield the title compound.

MS 952 (M+1)⁺

### Example 4

### Compound #4 and Compound #5

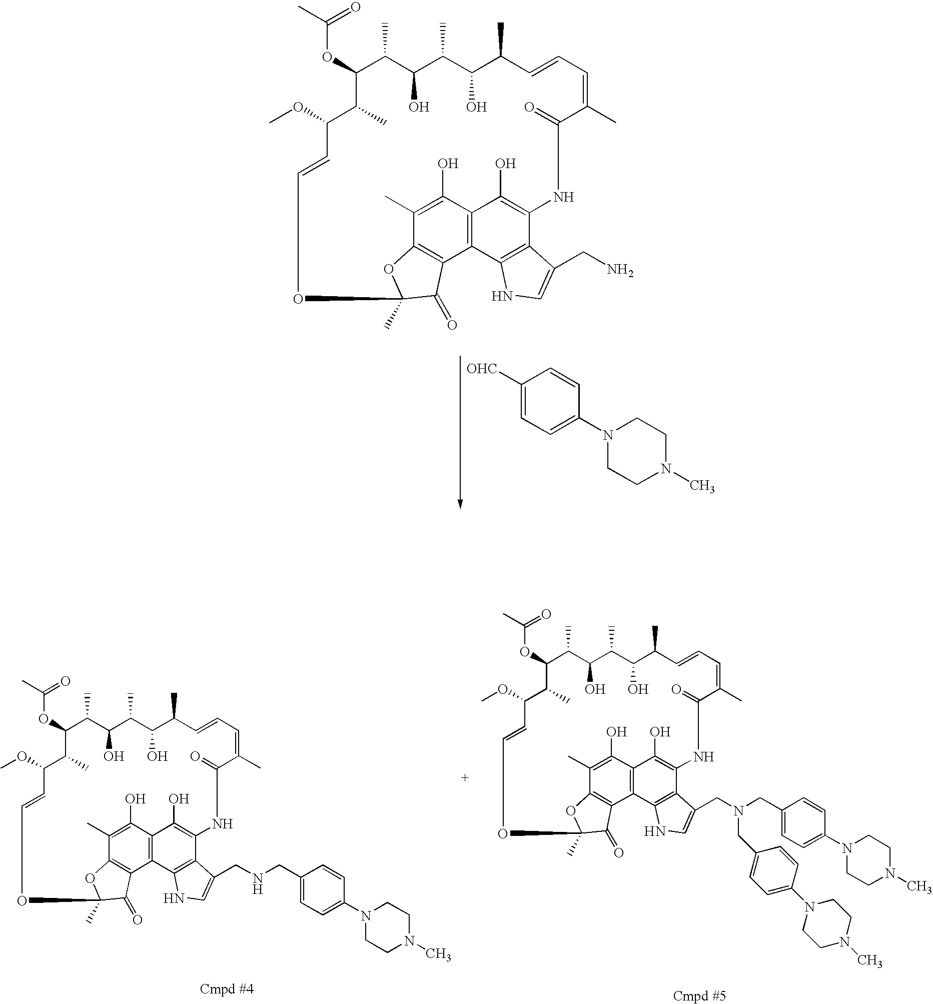

To a solution of Compound #1, prepared as in Example 1 (50 mg, 0.07 mmol) in DCE (2 mL) was added acetic acid (0.03 mL, 0.56 mmol), 4-(4-methyl-piperazin-1-yl)-benzaldehyde (68 mg, 0.33 mmol), and sodium triacetoxyborohydride (118 mg, 0.56 mmol). The resulting mixture was stirred at room temperature for 36 h, partitioned between saturated aqueous sodium bicarbonate and ethyl acetate, dried with sodium sulfate, and concentrated in vacuo. The resulting residue was purified by HPLC (C-18, MeCN/H₂O, gradient elution) to yield each of the title compounds (in separate fractions).

Compound #4: MS 938 (M+1)⁺

¹H NMR (300 MHz, CDCl₃): δ 13.58 (s, 1H), 13.22 (s, 1H), 12.49 (s, 1H), 7.13 (d, 1H), 7.11 (d, 2H), 6.90 (d, 2H), 6.89 (d, XH), 6.33 (dd, 1H), 6.20-6.16 (m, 2H), 5.91 (dd, 1H), 5.03 (dd, 1H), 4.81 (d, 1H), 4.02-3.36 (m, 9H), 3.22-3.19 (m, 4H), 3.01 (s, 3H), 2.94-2.91 (m, 1H), 2.59-2.57 (m, 4H), 2.42-2.38 (m, 1H), 2.36 (s, 3H), 2.29 (s, 3H), 2.06 (s, 3H), 2.02 (s, 3H), 1.83 (s, 3H), 1.28-1.22 (m, 1H), 0.99 (d, 3H), 0.89 (d, 3H), 0.23 (d, 3H), and −0.7 (d, 3H).

Compound #5: MS 1126 (M+1)⁺

### Example 5

### Biological Activity

The compounds of the present invention possess antibacterial activity and are therefore useful as antibacterial agents for the treatment of bacterial infections in humans and animals.

Minimum inhibitory concentration (MIC) is an indicator of in vitro antibacterial activity. The MIC is the lowest concentration of test compound that completely inhibits growth of the test organism. The in vitro antimicrobial activity of representative compounds of the present invention was determined by the microdilution broth method following the test method from the National Committee for Clinical Laboratory Standards (NCCLS). This method is described in the NCCLS Document M7-A4, Vol. 17, No. 2, “Methods for Dilution Antimicrobial Susceptibility Test for Bacteria that Grow Aerobically—Fourth Edition”, which is incorporated herein by reference.

In this method two-fold serial dilutions of test compound in cation adjusted Mueller-Hinton broth were added to wells in microdilution trays. The test organisms were prepared by adjusting the turbidity of actively growing broth cultures so that the final concentration of test organism after it is added to the wells was approximately 5×10⁴ CFU/well. Following inoculation of the microdilution trays, the trays were incubated at 35° C. for 16-20 hours and then read. The amount of growth in the wells containing the test compound was compared with the amount of growth in the growth-control wells (no test compound) used in each tray.

Representative compounds of the present invention were tested against a variety of pathogenic bacteria with results as listed in Table 2, below.

TABLE 2
In vitro Antibacterial Activity (MIC in μg/mL)

MIC (μg/mL)

| ID No. | A Strain | B Strain | C Strain | D Strain |
|---|---|---|---|---|
| 1 | <1 | 0.25 | 0.12 | 0.25 |
| 2 | 8 | 4 | 4 | 4 |
| 3 | 2 | 1 | 1 | 1 |
| 4 | 1 | 0.5 | 1 | 0.5 |
| 5 | 1 | 0.25 | 0.25 | 0.25 |

A: Enterococcus faecium OC 3312 vancomycin resistant;
B: Methicillin-resistant Staphylococcus aureus OC 3726 COL;
C: Staphylococcus aureus OC4172;
D: Methicillin-resistant Staphylococcus aureus OC 2878.

### Example 6

### Oral Formulation—Prophetic Example

As a specific embodiment of an oral composition, 100 mg of the Compound #5 prepared as in Example 4 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A compound of formula (I) 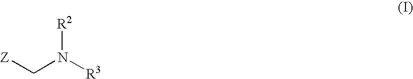
wherein
Z is selected from the group consisting of 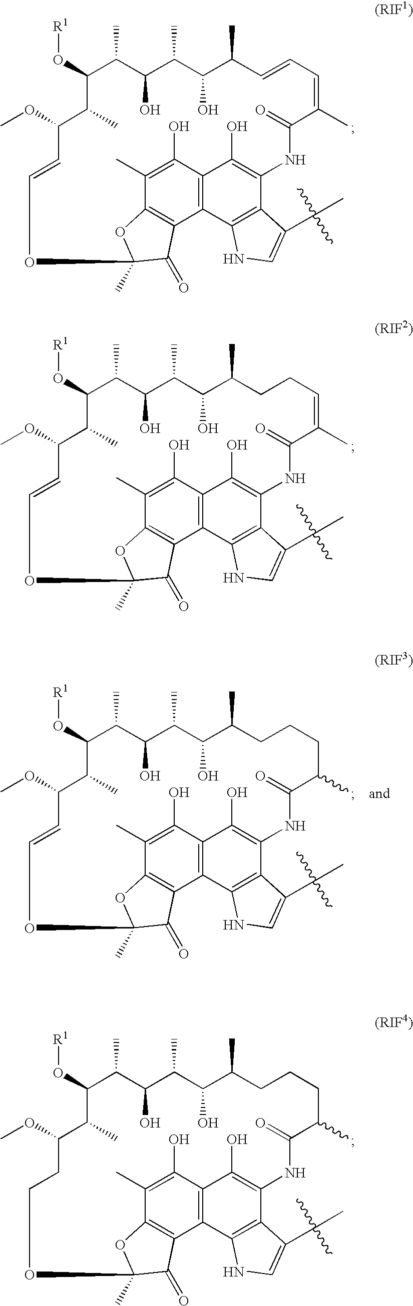
R¹ is selected from the group consisting of hydrogen and acyl;
R² and R³ are each independently selected from the group consisting of hydrogen, C₁₋₄alkyl and —(C₁₋₄alkyl)-(Ring A)-(Ring B);
wherein (Ring A) is selected from the group consisting of aryl and heteroaryl; wherein the aryl or heteroaryl is optionally substituted with one or more substituents independently selected from the group consisting of halogen, C₁₋₄alkyl, C₁₋₄alkoxy, halogenated C₁₋₄alkyl and halogenated C₁₋₄alkoxy;
wherein (Ring B) is selected from the group consisting of aryl, heteroaryl and heterocycloalkyl; wherein the aryl, heteroaryl or heterocycloalkyl is optionally substituted with one or more substituents independently selected from the group consisting of halogen, C₁₋₄alkyl, C₁₋₄alkoxy, halogenated C₁₋₄alkyl and halogenated C₁₋₄alkoxy;
alternatively R² and R³ are taken together with the nitrogen atom to which they are bound to form a 5 to 7 membered, saturated, nitrogen containing ring; wherein the 5 to 7 membered, saturated nitrogen containing ring is optionally substituted with one or more substituents independently selected from the group consisting of halogen, C₁₋₄alkyl, C₁₋₄alkoxy, halogenated C₁₋₄alkyl and halogenated C₁₋₄alkoxy;
or a pharmaceutically acceptable salt thereof.

2. A compound as in claim 1, wherein
Z is (RIF¹);
R¹ is selected from the group consisting of hydrogen and acyl;
R² and R³ are each independently selected from the group consisting of hydrogen, C₁₋₄alkyl and —(C₁₋₄alkyl)-(Ring A)-(Ring B);
wherein (Ring A) is selected from the group consisting of phenyl and 5 to 6 membered heteroaryl; wherein the aryl or 5 to 6 membered heteroaryl is optionally substituted with one to three substituents independently selected from the group consisting of halogen, C₁₋₄alkyl, C₁₋₄alkoxy, halogenated C₁₋₄alkyl and halogenated C₁₋₄alkoxy;
wherein (Ring B) is selected from the group consisting of phenyl, 5 to 6 membered heteroaryl and 5 to 6 membered heterocycloalkyl; wherein the phenyl, 5 to 6 membered heteroaryl or 5 to 6 membered heterocycloalkyl is optionally substituted with one to three substituents independently selected from the group consisting of halogen, C₁₋₄alkyl, C₁₋₄alkoxy, halogenated C₁₋₄alkyl and halogenated C₁₋₄alkoxy;
alternatively R² and R³ are taken together with the nitrogen atom to which they are bound to form a 5 to 6 membered, saturated, nitrogen containing ring; wherein the 5 to 6 membered, saturated nitrogen containing ring is optionally substituted with one to three substituents independently selected from the group consisting of halogen, C₁₋₄alkyl, C₁₋₄alkoxy, halogenated C₁₋₄alkyl and halogenated C₁₋₄alkoxy;
or a pharmaceutically acceptable salt thereof.

3. A compound as in claim 2, wherein
wherein
Z is (RIF¹);
R¹ is acyl;
R² and R³ are each independently selected from the group consisting of hydrogen, C₁₋₂alkyl and —(C₁₋₄alkyl)-(Ring A)-(Ring B);
wherein (Ring A) is phenyl; and wherein (Ring B) is selected from the group consisting of phenyl, 5 to 6 membered heteroaryl and 5 to 6 membered heterocycloalkyl; wherein the phenyl, 5 to 6 membered heteroaryl or 5 to 6 membered heterocycloalkyl is optionally substituted with one to two substituents independently selected from the group consisting of halogen, C₁₋₄alkyl, C₁₋₄alkoxy, fluorinated C₁₋₂alkyl and fluorinated C₁₋₂alkoxy;
alternatively R² and R³ are taken together with the nitrogen atom to which they are bound to form a 5 to 6 membered, saturated, nitrogen containing ring; wherein the 5 to 6 membered, saturated nitrogen containing ring is optionally substituted with one to two substituents independently selected from the group consisting of halogen, C₁₋₄alkyl, C₁₋₄alkoxy, fluorinated C₁₋₂alkyl and fluorinated C₁₋₂alkoxy;
or a pharmaceutically acceptable salt thereof.

4. A compound as in claim 3, wherein
wherein
Z is (RIF¹);
R¹ is —C(O)CH₃;
R² and R³ are each independently selected from the group consisting of hydrogen, methyl and 4-(4-methyl-piperazinyl)-benzyl;
alternatively, R² and R³ are taken together with the nitrogen atom to which they are bound to form 1-piperidinyl;
or a pharmaceutically acceptable salt thereof.

5. A compound as in claim 4, wherein
Z is (RIF¹);
R¹ is —C(O)CH₃;
R² and R³ are each independently selected from the group consisting of hydrogen and 4-(4-methyl-piperazinyl)-benzyl;
or a pharmaceutically acceptable salt thereof.

6. A pharmaceutical composition comprising a pharmaceutically acceptable carrier and a compound of claim 1.

7. A pharmaceutical composition made by mixing a compound of claim 1 and a pharmaceutically acceptable carrier.

8. A process for making a pharmaceutical composition comprising mixing a compound of claim 1 and a pharmaceutically acceptable carrier.

9. A method of treating a subject having a condition caused by or contributed to by bacterial infection, comprising administering to a subject in need thereof a therapeutically effective amount of the compound as in claim 1.

10. A method of preventing a subject from suffering from a condition caused by or contributed to by bacterial infection, comprising administering to a subject in need thereof a prophylactically effective dose of a compound as in claim 1.

11. The use of a compound as in claim 1 for the preparation of a medicament for treating or preventing a condition caused by or contributed to by bacterial infection, in a subject in need thereof.
